(19) United States
(12) Patent Application Publication
(10) Pub. No.: US 2012/0301421 A1
(43) Pub. Date: Nov. 29, 2012

(54) Deodorizer Composition and Method

(76) Inventor: Robert Joseph Hecking, Cincinnati, OH (US)

(21) Appl. No.: 13/458,461

(22) Filed: Apr. 27, 2012

Related U.S. Application Data
(63) Continuation-in-part of application No. 13/200,760, filed on Sep. 28, 2009.

(51) Int. Cl. A61L 9/01; A01P 1/00

(52) U.S. Cl. 424/76.8; 424/76.1

(57) ABSTRACT

The present invention relates to deodorizing compositions. The invention also relates to deodorizing the interior of the ventilation systems. The invention also relates to deodorizing the interior of vehicle ventilation systems and distributes fresh scent when fan in system is turned on for either heat or air conditioning. The present invention also encompasses a process for deodorizing the interior of a vehicle. The invention also relates to processes for deodorizing the interior of the vehicle ventilation systems and distributes fresh scent when fan in system is turned on for either heat or air conditioning.

### CROSS-REFERENCE TO RELATED APPLICATIONS

The present application is a continuation-in-part to and claims priority to nonprovisional U.S. patent application Ser. No. 13/200,760, filed Jul. 1, 2009, which was erroneously assigned Ser. No. 29/200,760 and which application is herein incorporated by reference in its entirety for all purposes.

### BACKGROUND ON THE INVENTION

1. Field of the Invention

The present invention relates to deodorizing compositions. The invention also relates to devices, methods and compositions for deodorizing ventilation systems. The invention also relates to deodorizing the interior of vehicles and vehicle ventilation systems.

2. Description of the Related Art

Bad odors in cars have been primarily linked to food or beverage spills, pet or baby accidents and/or cigarette smoke absorbed by the fabrics in the car, such as the headliners, upholstery and the carpets. Odors in the car ventilation system have received a secondary importance, as experienced detailers focus on the odors in the car's interior. It has been observed that when smoke odors were completely neutralized in vehicles interiors, the smoke odor re-emerged when the ventilation system was engaged. It was then discovered that the car ventilation also system contributes to the retention of smoke and musty odors.

Long term smoking in homes, restaurants or vehicles results in an odiferous brown film of cigarette tar, nicotine and other smoke residues on walls, ceilings and furniture. Today, car ventilation systems are run almost constantly while driving either on Heat or Air Conditioning. Therefore, these films are deposited on home and vehicle ventilation system distribution hoses or rigid tubing (duct work). Small cars can have up to 6 feet of intake and return ductwork. Larger SUV's can have 15 to 25 feet of ductwork. Many vehicles have cabin filters that take incoming air from the hood vents and filter out particulate dust and some minor odors.

Prior to the present invention, rental car managers, rental car management and detailers tried to neutralize these odors by spraying odor counteractant sprays or perfumes into the cabin filters or the external air intake under the hood near the windshield. These sprays were absorbed by the filter substrate, and little if any odor neutralizer reached the odor causing films on the ventilation ductwork walls. For a small time period the scent in the odor neutralizer products semi covered the bad odors, but they returned when the small amount of perfume was exhausted in the cabin filter.

Several retail companies (such as Procter & Gamble, Dial Corp, Yankee Candle, Lalean Corp.) make scented sticks or clips that attach to the car vent outlets, which perfume the air but do not neutralize odors on duct walls.

### SUMMARY OF THE INVENTION

The present invention relates to compositions related to deodorizing methods and compositions. The invention also relates to methods and compositions for deodorizing the interior of the ventilation systems and distributing fresh scent when the ventilation fan in the system is turned on for either heat or air conditioning.

The present invention also encompasses a process for deodorizing the interior of a vehicle. The invention also relates to methods and compositions for deodorizing the interior of a vehicle ventilation systems and distributing fresh scent when the fan in system is turned on for either heat or air conditioning.

One embodiment of the present invention provides for odor eliminating compositions. In one embodiment, the aerosol compositions are aqueous based and include: (a) at least one odor neutralizing agent; (b) at least one adhesion agent; and (c) at least one dispersant.

Optionally, the composition further comprises one or more of the following: (d) at least one fragrance; (e) at least one buffering system; (f) at least one surfactant; and (g) at least one compressed gas propellant.

In one embodiment, the at least one odor neutralizing agent is selected from the group consisting of zeolites, activated carbons, zinc salts, zinc oxides, cyclodextrins, fatty alcohol esters, aliphatic aldehydes (C10, C11, and C12), zinc ricinoleate actives, benzaldehyde, soyethyl morpholinium ethosulfate, Meelium®, and Metazene®.

In one embodiment, the zinc salts are selected from the group consisting of zinc borate, zinc caprylate, zinc chloride, zinc ricinoleate, zinc sulfate heptahydrate, zinc undecylenate, and mixtures thereof.

In one embodiment, the composition is further combined with a least one further deodorizing active ingredient. In one embodiment, the at least one further deodorizing active ingredient is a quaternary ammonium compound.

In one embodiment, the at least one adhesion agent is selected from the group consisting of glycol (also known as a diol) and polyhydric alcohol (also known as a polyol, sugar alcohol or polyalcohol). A glycol is a molecule that contains two hydroxyl groups. In one embodiment, the at least one adhesion agent is a glycol selected from the group consisting of, propylene glycol, dipropylene glycol, butylene glycol, hexylene glycol, polyethylene glycol, and mixtures thereof. In one embodiment, the at least one adhesion agent is a polyhydric alcohol selected from the group consisting of inositol (6-carbon), glycol (2-carbon), glycerol (3-carbon), erythritol (4-carbon), threitol (4-carbon), arabitol (5-carbon), xylitol (5-carbon), ribitol (5-carbon), mannitol (6-carbon), sorbitol (6-carbon), dulcitol (6-carbon), fucitol (6-carbon), iditol (6-carbon), isomalt (12-carbon), maltitol (12-carbon), lactitol (12-carbon), and polyglycitol. In one embodiment, the at least one adhesion agent is a polyhydric alcohol selected from the group consisting of mannitol and sorbitol. Other agents include medium-chain triglycerides (MCTs), vegetable oils, and benzylated alcohol solvents e.g., benzyl alcohol.

In one embodiment, the at least one dispersant is selected from the group consisting of butyl ethers, dipropylene glycol methyl ether (DOWANOL™ DPM), D[propylene glycol methyl ether, ethyl alcohol SDA-40 and isopropyl alcohol.

In another embodiment, the at least one dispersant is selected from the group consisting of alcohols such as methyl alcohol, ethyl alcohol, n-propyl alcohol, isopropyl alcohol, n-butyl alcohol, sec-butyl alcohol, glycols such as ethylene glycol, diethylene glycol, triethylene glycol, propylene glycol; glycol ethers such as ethylene glycol monomethyl ether and triethylene glycol monoethyl ether; and any combination thereof.

The invention also provides for methods of deodorizing using compositions of the present invention.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 shows a schematic view of an embodiment of a car ventilation system utilized in the process of the present invention.

FIG. 2 shows a cross-sectional schematic of a dispensing device used in the present invention. The dispensing tube can be rigid and/or of various lengths depending upon application.

FIG. 3 shows a cross-sectional schematic of a dispensing device used in the present invention. The dispensing tube can be flexible and/or of various lengths depending upon application.

FIG. 4 shows a schematic of a prior art, dispensing device used with a ventilation vent.

FIG. 5 shows a schematic of a dispensing device of the present invention used for composition application within a ventilation vent.

It is to be emphasized that these figures illustrate but one embodiment of the auto ventilation system used in the present invention. Other structures can be used.

### DESCRIPTION OF THE REFERENCED NUMERALS

In reference to the drawings, similar reference characters denote similar elements throughout all the drawings. The following is a list of the reference characters and associated element:
1. Side ventilation outlet
2. Side defroster outlet
3. Center ventilator outlet
4. Floor outlet
5. Front defroster outlet
6. Heater unit
7. Air duct
8. Blower motor unit
9. Control lever
10. Defroster nozzle
11. Side demister duct
12. Center ventilator duct
13. Side ventilator duct
14. Side ventilator joint
15. Outside air
16. Inside air
100 Spray System
110 Spring tensioning means
120 Aerosol container
130 Cap
140 Odor control composition
150 Valve
160 Internal dip tube
170 Liquid odor control composition
180 Propellant
190 Discharge Tubing

### DETAILED DESCRIPTION

Before describing the present invention in detail, it is to be understood that this invention is not limited to particularly exemplified systems or process parameters as such may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference for all purposes and in their entirety to the same extent as if each individual publication, patent or patent application was specifically and individually indicated as incorporated by reference.

It must be noted that, as used in this specification and the appended claims, the singular forms “a,” “an” and “the” include plural referents unless the content clearly dictates otherwise. Thus, for example, reference to a “surfactant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

As will be appreciated by one having ordinary skill in the art, the methods and compositions of the invention substantially reduce or eliminate the disadvantages and drawbacks associated with prior art methods and compositions.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As used in the present specification, the following words and phrases are generally intended to have the meanings as set forth below, except to the extent that the context in which they are used indicates otherwise.

In this specification, various terms are defined as follows:

The term “effective amount” means a level of an ingredient sufficient to affect the purpose of the ingredient. For illustration, an “effective amount” of an odor-controlling composition would mean an amount sufficient to absorb odor and/or neutralize odor to the point that it is less noticeable. For further illustration, an “effective amount” of a preservative would be an amount of the preservative sufficient to prevent spoilage, or prevent microorganism growth in the composition.

The term “odor eliminating” means the removal of odors. The term “eliminating”, in the context of odors, is defined as lessening the intensity, duration, and offensiveness of an odor or smell such that the odor or smell is at least marginally eliminated.

The term “odor reduction” means the reduction of odors.

The term “controlling odor” means reducing and/or eliminating odor.

The term “removing”, in the context of odors, is herein defined as eliminating or ending the intensity, duration, and offensiveness of an odor or smell.

Unless otherwise defined, all technical and scientific terms used have the same meaning as commonly understood by one of ordinary skill in the art to which this invention pertains. Although methods and materials similar or equivalent to those described herein can be used in the practice and testing of this invention, suitable methods and materials are described below. In addition, the materials, methods, and examples are illustrative only and not intended to be limiting.

Embodiments of this invention include compositions and methods for reducing or eliminating odors emitted by substrate. A substrate that can be treated by embodiments of this invention include smoke, fecal matter, vomit, urine, or sewage sludge, which can create environmental malodors.

The present invention provides for odor eliminating compositions. In one embodiment, the aerosol compositions are aqueous based and include: (a) at least one odor neutralizing agent; (b) at least one adhesion agent; and (c) at least one dispersant.

Optionally, the composition further comprises one or more of the following: (d) at least one fragrance; (e) at least one buffering system; (f) at least one surfactant; and (g) at least one compressed gas propellant.

In one embodiment, the at least one odor neutralizing agent is selected from the group consisting of zeolites, activated carbons, zinc salts, zinc oxides, cyclodextrins, fatty alcohol esters, aliphatic aldehydes (C10, C11, and C12), zinc ricinoleate actives, benzaldehyde, soyethyl morpholinium ethosulfate, Meelium®, and Metazene®.

In one embodiment, the composition further comprises one or more of metal salts of an unsaturated hydroxy carboxylic acid.

In one embodiment, the metallic salts are water-soluble zinc salts, copper salts or mixtures thereof, and more preferably zinc salts, especially zinc ricinoieate. These salts are preferably present in the present invention as an odor control agent primarily to absorb amine and sulfur-containing compounds. Low molecular weight sulfur-containing materials, e.g., sulfide and mercaptans, are components of many types of malodors, e.g., food odors (garlic, onion), body/perspiration odor, breath odor, etc. Low molecular weight amines are also components of many malodors, e.g., food odors, body odors, urine, etc.

In one embodiment, the zinc salts are selected from the group consisting of zinc borate, zinc caprylate, zinc chloride, zinc ricinoleate, zinc sulfate heptahydrate, zinc undecylenate, and mixtures thereof.

In one embodiment, the zinc salts are zinc salts of ricinoleic acid. In another embodiment, the zinc ricinoleate is combined with at least one alkoxylated amine. In another embodiment, the zinc ricinolate is Grillocin® or Tego-Sorb®.

Zinc ricinoleate is largely insoluble in water and therefore has to be used in combination with solvents and solubility promoters in order to obtain effective preparations. The solvents used are in most cases mono- or polyhydric alcohols, optionally with the addition of water.

Grillocin® contains a clearly defined substance, zinc ricinoleate, synergistically combined with other zinc compounds made up of multiple hydroxylated sebatic acids, oxamines and resinic acids.

Tego-Sorb® is zinc ricinoleate with solubilizers and/or benzalkonium chloride. In some embodiments, the ricinoleate is in combination with one or more substituted monocyclic organic compounds.

In one embodiment, the composition is further combined with a least one further deodorizing active ingredient. In one embodiment, the at least one further deodorizing active ingredient is a quaternary ammonium compound.

In one embodiment, the at least one adhesion agent is selected from the group consisting of glycol (also known as a diol) and polyhydric alcohol (also known as a polyol, sugar alcohol or polyalcohol). A glycol is a molecule that contains two hydroxyl groups. In one embodiment, the at least one adhesion agent is a glycol selected from the group consisting of, propylene glycol, dipropylene glycol, butylene glycol, hexylene glycol, polyethylene glycol, and mixtures thereof.

In one embodiment, the at least one dispersant is selected from the group consisting of butyl ethers, Dipropylene Glycol Methyl Ether (DOWANOL™ DPM), D[propylene glycol methyl ether, ethyl alcohol SDA-40, isopropyl alcohol, tropropylene, glycol (mono) methyl ether, alkylene glycol and alkyl polyglycoside.

Other agents include medium-chain triglycerides (MCTs), vegetable oils, animal oils and microalgae oils.

Medium-chain triglycerides (MCTs) are medium-chain (6 to 12 carbons) fatty acid esters of glycerol including caproic acid (C6), caprylic acid (C8), capric acid (C10) and lauric acid (C12). MCTs are composed of a glycerol backbone and three fatty acids, wherein one or more of the fatty acid chains attached to the glycerol are medium chain in length.

In one embodiment, the at least one dispersant is selected from the group consisting of butyl ethers, diethylene monethyl ether—(carbitol), D[propylene glycol methyl ether, ethyl alcohol SDA-40, isopropyl alcohol, tropropylene, glycol (mono) methyl ether, alkylene glycol, alkyl polyglycoside.

In another embodiment, the at least one dispersant is selected from the group consisting of alcohols such as methyl alcohol, ethyl alcohol, n-propyl alcohol, isopropyl alcohol; glycol ethers such as ethylene glycol monomethyl ether and triethylene glycol monoethyl ether; and any combination thereof.

Meelium is a blend of polyhydroaromatic sulfonates. Neutrolair D-7 is a mixture of geranyl crotonalte and dihexyl fumarate, Vandor B is principally 3,5,5-trimethylhexanal. Forestall is soyaethyl morpholinium ethosulfate.

### Additional Odor-Reacting Compounds

Odor-reacting compounds are an important feature of the compositions described herein. Ammonia, amines, and thiol compounds are common odorants found in urine, vomit, and other organic contaminants. Odor-reacting compounds are those that are capable of chemically reacting with one or more of these odorants, thereby reducing or eliminating these odors. Preferably, odor-reacting compounds are selected from those compounds that do not inherently have strong odors or aromas and those that are not used as perfumes, fragrances, or aromas. Odor-reacting compounds suitable for use in the liquid or powder compositions described herein include aldehyde compounds, formaldehyde-donating compounds, ketones, and oxidizing agents.

Aldehyde compounds can react with odorous amine compounds to form an imine structure. Aldehyde compounds can also react with thiol compounds to form a thioacetal structure. Formaldehyde-donor compounds, which have similar reactivity with amines and thiols, can be used in combination or interchangeably with aldehyde compounds. The reaction of odorous amines and thiols with either the aldehyde compound or the formaldehyde-donor compound results in the products of imine and thioacetal, both of which are larger molecules than their odorous substituents. As such, these resulting structures are less volatile than their predecessors and have little to no smell.

In another embodiment, “odor Mockers” can be used as an optional odor control agent to mitigate the effects of malodors. In order to be effective, the odor Mockers normally have to be present at all times. If the odor Mocker evaporates before the source of the odor is gone, it is less likely to control the odor. Also, the odor Mockers can tend to adversely affect aesthetics by blocking desirable odors like perfumes. Odor Mockers are disclosed in more detail in U.S. Pat. Nos. 4,009,253; 4,187,251; 4,719,105; 5,441,727; and 5,861,371.

In another embodiment, reactive aldehydes can be used as an odor control agent to mitigate the effects of malodors. Non-limiting examples of suitable reactive aldehydes include Class I aldehydes, Class II aldehydes, and mixtures thereof. Non-limiting examples of Class I aldehydes include anisic aldehyde, o-allyl-vanillin, benzaldehyde, cuminic aldehyde, ethyl-aubepin, ethyl-vanillin, heliotropin, tolyl aldehyde, and vanillin. Non-limiting examples of Class II aldehydes include 3-(4′-tert-butylphenyl)propanal, 2-methyl-3-(4′-tert-butylphenyl)propanal, 2-methyl-3-(4′-isopropylphenyl)-propanal, 2,2-dimethyl-3-(4-ethylphenyl)propanal, cinnamic aldehyde, α-amyl-cinnamic aldehyde, and α-hexyl-cinnamic aldehyde. These reactive aldehydes are described in more detail in U.S. Pat. No. 5,676,163.

Reactive aldehydes, when used, can include a combination of at least two aldehydes, with one aldehyde being selected from acyclic aliphatic aldehydes, non-terpenic aliphatic aldehydes, non-terpenic alicyclic aldehydes, terpenic aldehydes, aliphatic aldehydes substituted by an aromatic group and bifunctional aldehydes; and the second aldehyde being selected from aldehydes possessing an unsaturation alpha to the aldehyde function conjugated with an aromatic ring, and aldehydes in which the aldehyde group is on an aromatic ring. This combination of at least two aldehydes is described in more detail in International Patent Application Pub. No. WO 00/49120.

As used herein, the term “reactive aldehydes” further encompasses deodorizing materials that are the reaction products of (i) an aldehyde with an alcohol, (ii) a ketone with an alcohol, or (iii) an aldehyde with the same or different aldehydes. Such deodorizing materials can be: (a) an acetal or hemiacetal produced by means of reacting an aldehyde with a carbinol; (b) a ketal or hemiketal produced by means of reacting a ketone with a carbinol; (c) a cyclic triacetal or a mixed cyclic triacetal of at least two aldehydes, or a mixture of any of these acetals, hemiacetals, ketals, hemiketals, or cyclic triacetals. These deodorizing perfume materials are described in more detail in International Patent Application Pub. No. WO 01/07095.

In another embodiment, flavanoids can be used as an odor control agent. Flavanoids are compounds based on the C6-C3-C6 flavan skeleton. Flavanoids can be found in typical essential oils. Such oils include essential oil extracted by dry distillation from needle leaf trees and grasses such as cedar, Japanese cypress, eucalyptus, Japanese red pine, dandelion, low striped bamboo and cranesbill and can contain terpenic material such as alpha-pinene, beta-pinene, myrcene, phencone and camphene. Also included are extracts from tea leaf. Descriptions of such materials can be found in JP 02284997 and JP 04030855.

In one embodiment, the composition comprises zeolites. In one embodiment, the zeolites are “intermediate” silicate/aluminate zeolites. The intermediate zeolites are characterized by SiO₂/AlO₂ molar ratios of less than about 10. Preferably the molar ratio of SiO₂/AlO₂ ranges from about 2 to about 10. The intermediate zeolites can have an advantage over the “high” zeolites. The intermediate zeolites have a higher affinity for amine-type odors, they are more weight efficient for odor absorption because they have a larger surface area, and they are more moisture tolerant and retain more of their odor absorbing capacity in water than the high zeolites. A wide variety of intermediate zeolites suitable for use herein are commercially available as Valor CP300-68, Valfor 300-63, Valfor CP300-35, and Valor CP300-56, available from PQ Corporation, and the CBV100 series of zeolites from Conteka. Zeolite materials marketed under the trade name Abscents and Smellrite, available from The Union Carbide Corporation and UOP may be used.

When zeolites are used as odor control agents in compositions that are to be sprayed onto surfaces, the zeolite material preferably has a particle size of less than about 10 microns and is present in the composition at a level of less than about 1% by weight of the composition.

In one embodiment, conventional surfactants including anionic surfactants, ionic surfactants, amphoteric surfactants, zwitterionic surfactants or any combinations or mixtures thereof may be employed.

In additional to surfactants, the diluted water-soluble concentrate of the present invention may be used with conventional solvents, dyes, preservatives, emulsifying agents, perfumes, antibacterial agents, thickeners, conditioners, antistatic agents, silicone surfactants, and other like ingredients that are typically present in conventional home care formulations. Mixtures and/or combinations of the aforementioned additional formulating agents may also be employed in the present invention. The amounts of the additional formulating agents that may be employed in the present invention are within ranges that are well known to those skilled in the art and further formulating is performed using processes that are also well known in the art.

In one embodiment, the composition further comprises one or more antioxidant. In one embodiment, the one or more antioxidant is selected from one or more of a group consisting of ascorbic acid and salts thereof, tocopherol and its derivatives, butyl hydroxyanisole (BHA), butyl hydroxytoluene (BHT), erythorbic acid, propyl gallate, sodium erythorbate, tertiary butyl hydroquinone (TBHQ) and rosemary.

A co-solvent may be optionally included in the aerosol composition to assist the solubilization of the ingredients therein or assist the formation of a desired emulsion. The co-solvent is preferably a low molecular weight monohydric alcohol, such as a C1-C3 alcohol including methanol, ethanol, propanol, and isopropanol. Additionally, the co-solvent may also include other low molecular weight organic solvent such as acetone. In one embodiment, the co-solvent is ethanol. In another embodiment, the co-solvent is isopropanol.

As a co-solvent may contribute to the total VOC content of the composition, the presence of a co-solvent in the aerosol composition is optional and preferably in amount of no more than about 40 wt %, and preferably at a concentration lower than the water content. In a preferred embodiment, no solvent is present and, thus, an emulsifier is required to be present in an amount of from about 0.4 to about 4 wt % to insure formation of the desired emulsion. On the other hand, the emulsifier content may be reduced if a co-solvent is utilized. In one embodiment, the air treating composition does not include any emulsifier.

In one embodiment, the aerosol compositions have a volatile organic content (VOC) of less than 5.0% as to the total composition based on 100 wt. %. In one embodiment, the VOC content is less than 3.0% as to the total composition. In one embodiment, the VOC content is less than 1.0%.

The pH of the composition should fall in the range of from about 6 to about 9, more preferably in the range of from about 6 to about 8. The amount of pH adjusting agent included in the air treating composition to obtain the desired pH would be apparent to those of ordinary skill in the art. Preferably, the amount of pH modifying agent may be present in an amount of from about 0 to about 5 wt %, more preferably from about 0 to about 2 wt %.

In one embodiment, the at least one odor neutralizing agent comprises at least one alkylene glycol. In one embodiment, the at least one alkylene glycol is included in an amount of about 0.1 to about 10 wt. % of the composition, preferably about 0.1 to about 5 wt. %, and most preferably about 0.5 to about 2 wt. %. In one embodiment, the at least one alkylene glycol is selected from the group consisting of mono-, di- or tri-alkylene glycols, such as for example triethylene glycol, propylene glycol, dipropylene glycol, and mixtures thereof. In one embodiment, the mixture is about 0.1 to about 30 wt. % triethylene glycol and about 1 to about 10 wt. % propylene glycol.

In one embodiment, the at least one surfactant is included in an amount of about 0.1 to about 10 wt. % of the composition, preferably about 0.1 to about 5 wt. %. The surfactant serves as a solubilizer alone or in conjunction with the alkylene glycol component for the fragrance, preservative or other component, which may be present which is not soluble in the aqueous carrier of the composition. Surfactants otherwise suitable for use can be nonionic, anionic, amphoteric, cationic, or a combination thereof.

Nonionic surfactants suitable for use in the compositions of the present invention include alkoxylated compounds such as, for example, fatty alcohol alkoxylates, alkoxylated polyol fatty acid esters; alkyl polyglucosides; alkanolamides, including fatty acid alkanolamides; fatty alcohol polyalkyleneglycol ethers; oxo-alcohol polyalkylene glycol ethers; alkylphenol polyalkylene glycol ethers; fatty alcohol poly-poly glycol ethers (e.g. ethylene oxide/propylene oxide adducts); and alkyl dimethyl amine oxide, as well as nonionic polymers such as for example ethylene oxide/propylene oxide block copolymers. The alkoxy component is preferably ethoxy.

Anionic surfactants suitable for use in the compositions of the present invention include soaps, alkyl benzene sulfonates, alkane sulfonates, α-olefin sulfonates; α-sulfo fatty acid methyl esters; fatty alcohol sulfates or alkyl sulfates; alkyl ether sulfates including fatty alcohol ether sulfates and oxo-alcohol ether sulfates; and the like, as well as combinations thereof.

Examples of the quaternaries include oleyl di(polyoxyethylene)methyl ammonium chloride, stearyl dimethyl benzyl ammonium chloride, distearyldimethylanunonium chloride, stearyl trimethyl ammonium chloride, stearyl tri(polyoxyethylene)ammonium chloride, polyoxypropylene methyl diethyl ammonium chloride, myristyl dimethyl benzyl ammonium chloride, lauryl trimethyl ammonium chloride, etc.

Examples of the amphoteric surfactant include 2-alkyl-N-carboxymethyl-N-hydroxyethyl imidazolinium betaine, sodium undecyl hydroxyethyl imidazolinium betaine, undecyl-N-hydroxyethyl-N-carboxymethyl imidazolinium betaine, stearyl dihydroxyethyl betaine, coconut oil fatty acid amidopropyl betaine, sodium coconut oil alkyl-N-carboxyethyl-N-hydroxyethyl imidazolinium betaine, disodium coconut oil alkyl-N-carboxymethoxyethyl-N-carboxymethyl imidazolinium lauryl sulfate, N-coconut oil fatty acid acyl-L-arginine ethyl-DL-pyrrolidone-carboxylate, etc.

In one embodiment, the compressed gas is included in an amount of about 5 to about 70 wt. %, preferably about 10 to about 60 wt. %, and most preferably about 20 to about 50 wt. %. The compressed gas propellant is useful in the stated amounts for providing a suitable particle size and spray rate over the life of the product when the pressure within the container is sufficient to provide an acceptable spray.

Compressed gases suitable for use in the compositions of the present invention include are, for example, nitrogen, air, carbon dioxide, nitrous oxide, inert gases, and mixtures thereof. In one embodiment, the propellants useful in the present invention are non-hydrocarbons.

In one embodiment, the fragrance is present in an amount ranging from about 0.01 to about 10 wt. %, more preferably from about 0.01 to about 5 wt. %, and most preferably from about 0.01 to about 3 wt. %. In one embodiment, the fragrance may comprise one or more fragrant materials or materials that provide chemically active vapors. In one embodiment, the fragrance can comprise and/or include volatile, fragrant compounds including, but not limited to natural botanic extracts, essences, fragrance oils, synthetic fragrant materials and so forth. Exemplary oils and extracts include, but are not limited to, those derived from the following plants: almond, amyris, anise, armoise, bergamot, cabreuva, calendula, canaga, cedar, chamomile, coconut, eucalyptus, fennel, jasmine, juniper, lavender, lemon, orange, palm, peppermint, quassia, rosemary, spearmint, thyme, and so forth.

The compositions of the present invention may comprise additional additives in any combination thereof, as long as the effect of the deodorizer against odor of the present invention is maintained.

In one embodiment, the compositions of the present invention may comprise one or more rust inhibitor for aerosol cans. In one embodiment, the one or more rust inhibitor may be diethanol amine, isopropylamine, cyclohexylaminne, sodium benzoate, dibutoxyethylacid phosphate, c12-c14 alkyammoniumcarboxylate, or other rust inhibitors that may be used if they do not interfere with the action of the compositions.

Compositions of the present invention may be also be sprayed directly onto the target surface and therefore may be packaged in a spray dispenser. The spray dispenser can be any of the manually activated means for producing a spray of liquid droplets as is known in the art, e.g. trigger-type, pump-type, electrical spray, hydraulic nozzle, sonic nebulizer, high pressure fog nozzle, non-aerosol sell-pressurized, and aerosol-type spray means. Automatic activated means can also be used herein. These types of automatic means are similar to manually activated means with the exception that a compressor replaces the propellant.

The spray dispenser can be an aerosol dispenser. The aerosol dispenser comprises a container that can be constructed of any of the conventional materials employed in fabricating aerosol containers. The dispenser must be capable of withstanding internal pressure in the range of from about 5 to about 120 p.s.i.g. or alternatively from about m to about 100 p.s.i.g. The one important requirement concerning the dispenser is that it be provided with a valve member that will permit the inventive compositions contained in the dispenser to be dispensed in the form of a continuous stream or continuous spray of droplets. The aerosol dispenser utilizes a pressurized sealed container from which the inventive composition is dispensed through an actuator/valve assembly under pressure. In one embodiment, incorporating a gaseous component generally known as a propellant pressurizes the aerosol dispenser. In one embodiment, the propellant may be Dymel®134a/P (1,1,1,2 tetrafluorethane), a high-pressure, nonflammable, non-ozone-depleting fluorocarbon propellant intended as an alternative to CFCs.

In one embodiment, the valve may be fitted with a tube to inject formula into the ventilation system and propel the deodorizing composition deeper and through the entire car ventilation system.

Alternatively in one embodiment, the spray dispenser can be a sell-pressurized non-aerosol container having a convoluted liner and an elastomeric sleeve. The sell-pressurized dispenser comprises a liner/sleeve assembly containing a thin, flexible radially expandable convoluted plastic liner of from about 0.010 to about 0.020 inch thick, inside an essentially cylindrical elastomeric sleeve.

In general, the orifice size of the spray dispenser passageways, chambers, inlet and outlet orifices can be sized appropriately, which is to say generally enlarged in internal diameter with respect to sizes appropriate for thin liquids like water, to an extent governed by the viscosity of the inventive compositions to provide suitable dispensing characteristics.

In one embodiment, the invention provides for devices for use with the compositions disclosed above. In one embodiment, the device comprises a vessel capable of containing the composition of the present invention configured as an aerosol can, a pump spray or pressurized reservoir to be driven by compressed air driven through the valve into a delivery tube capable of delivering the product into crevasses.

In one embodiment, the invention provides for devices having a delivery tube that is from about 2 to about 36 inches or longer in length and a width from about 1/32″ inside diameter to ½″ inside diameter, which deliver product into ventilation system.

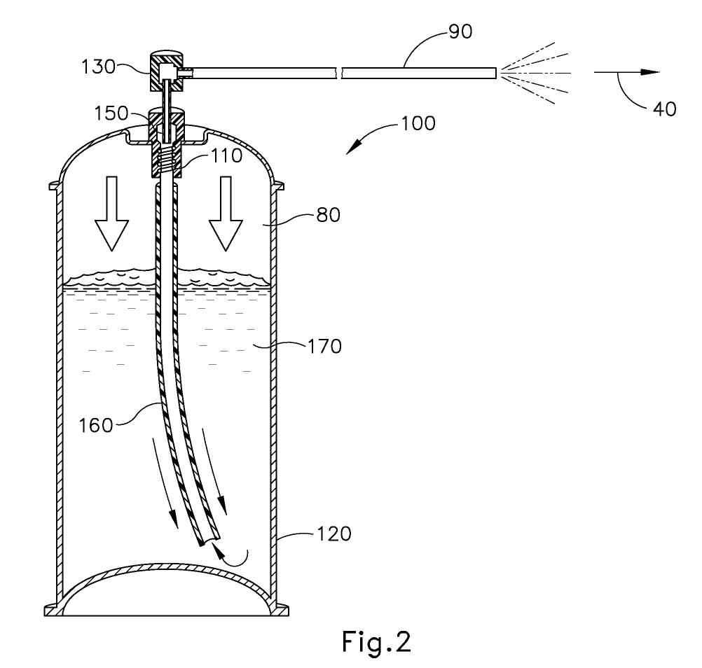

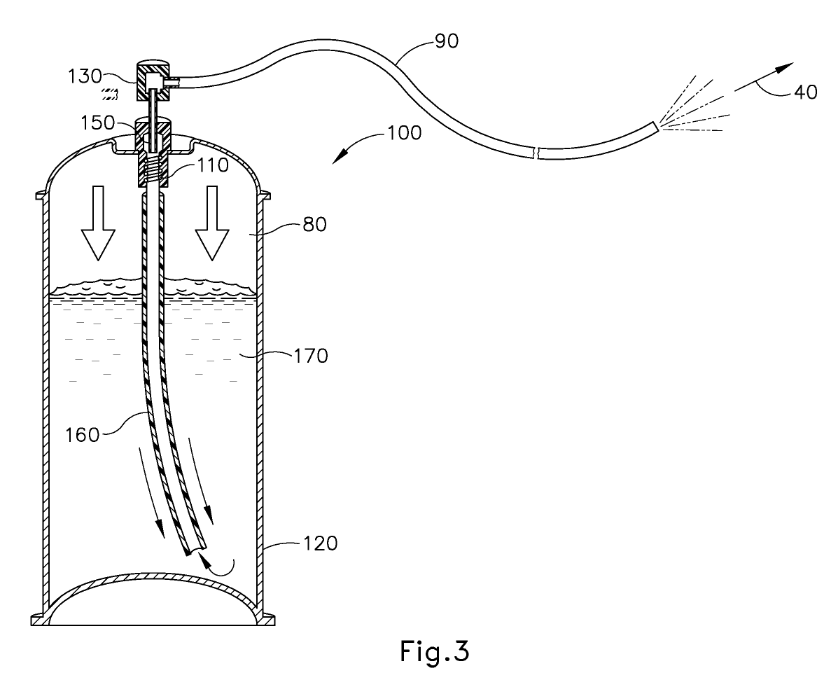

In one embodiment, the delivery device is a pressurized spray system 100 as shown in FIGS. 2 and 3, comprising a container 120 capable of holding an aerosol, an activation nozzle 130 which is in fluid communication with the interior volume of container 120 by a valve means 150 configured to open upon pressure induced by nozzle 130. The valve means 150 may further comprise a tensioning means 110, which is adapted for closing the valve 150 upon release of tension from nozzle 130. The container 120 is adapted for holding a liquid deodorizer composition 170 which is under pressure from compressed gas 80 configured to exert pressure on the liquid deodorizer composition 170 such that upon opening of valve 150, the liquid deodorizer composition 170 is forced through the valve 150 into nozzle 130. Nozzle 130 is adapted to comprise a tube 90 and is in fluid communication with tube 90 such that liquid deodorizer composition 170 forced through nozzle 130 is dispelled through tube 90 creating a discharge mist 140. The container 120 may further comprise an internal dip tube 160 for keeping the liquid deodorizer composition 170 in communication with valve 150.

In one embodiment, a suitable actuator and valve for use with the container may be the Kosmos 21-8142-0.016″ orifice; Gasket Stem spring 5030-10-1140; Super 90 Stem-0.018 orifice; Dip tube 09-2050-00-4 20/32″-dispensing tube; and Buna Gasket 05-5110-51, all from Precision Valve Corp., Rye Brook, N.Y.

As shown in FIG. 2, the tube 90 can be a rigid tube capable of maintaining its shape. As shown in FIG. 3, the tube 90 can be a flexible tube capable of adapting its shape to the contours of the ventilation system.

As shown in FIGS. 2 and 3, the length of the dispensing tube 90 can be of various lengths depending upon application.

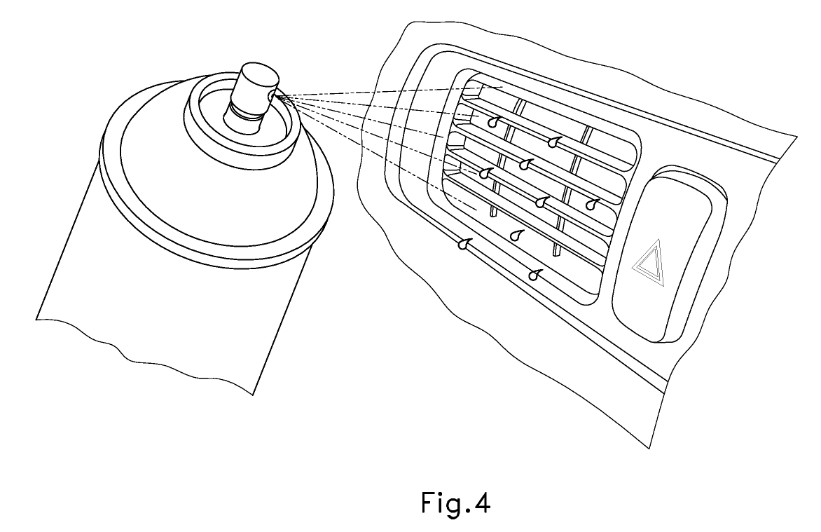

FIG. 4 shows a schematic of a prior art dispensing device used with a ventilation vent wherein the spray is dispersed onto a side ventilation outlet 1. Without dispensing tube 90, the spray does not reach the intended surfaces to be treated.

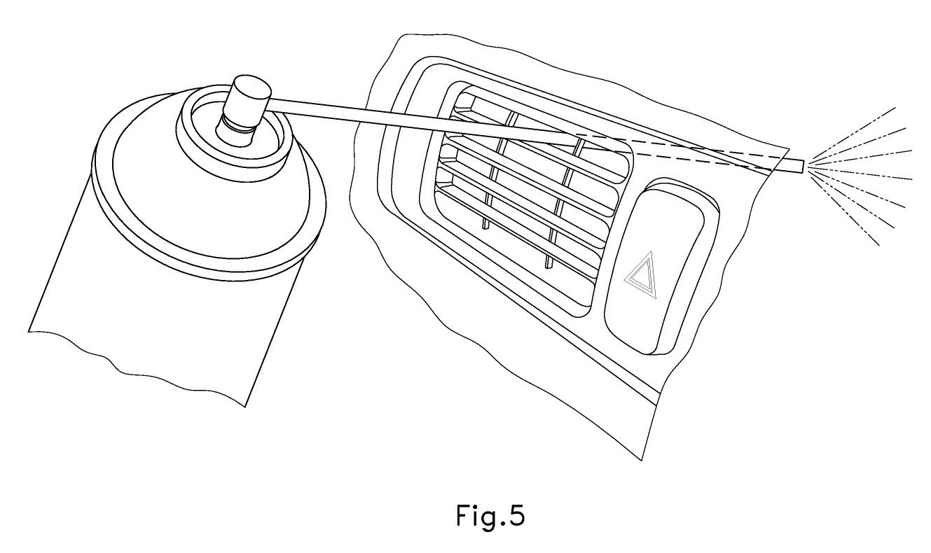

FIG. 5 shows a schematic of a dispensing device of the present invention used with a ventilation vent wherein the spray is dispersed through a side ventilation outlet 1. With dispensing tube 90, the spray is directed to distal portions of the ventilation system in order to reach the intended surfaces to be treated using composition of the present invention.

The present invention provides a method dispensing a liquid odor absorbing or neutralizing composition of the present invention by use of a delivery means such as a pressure propelled gas of glycol based product capable of precipitate odors on ventilation systems walls with a deodorizer to neutralize odors a scent to freshen. In one embodiment, the deodorizer is dispensed through a small diameter tube to reach into ventilation systems were odors are trapped. The invention can deliver and dispense a deodorizer agent (e.g. fragrance, perfume, deodorant, absorbent, odorant, perfume, deodorizer, or combination of thereof) to reach malodors and deodorize them in ventilation systems economically.

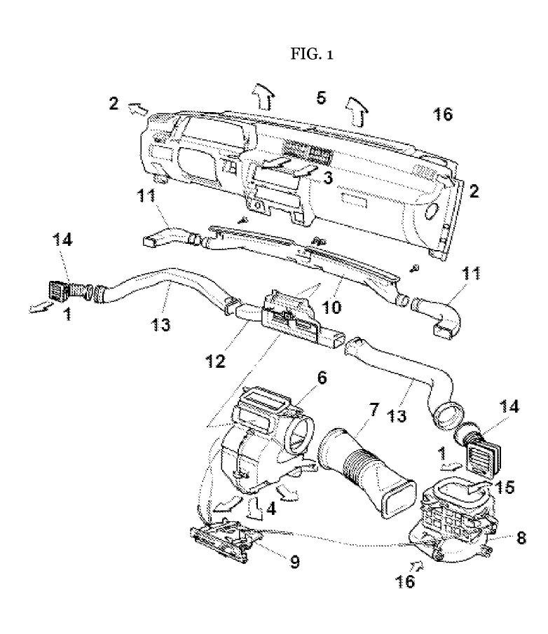

In one embodiment, the invention is used in a motor vehicle ventilation system as shown in FIG. 1. In one embodiment, the deodorizer is coated onto the inner surfaces of a motor vehicle ventilation system 16 by delivering the deodorizer in aerosol form though a tube. The tube not only avoids spraying product on vent louvers but also focuses the spray pattern so that it travels farther in the ventilation system.

In general, the front interior portion of a motor vehicle may include provisions for ventilation. In some embodiments, a front interior portion may include one or more vents. In some cases, the vents could be disposed on the dashboard. In other cases, the vents could be disposed at other portions of the front interior portion. For example, in some embodiments vents may be disposed on doors, under seats or in a central console of the front interior portion. Additionally, vents could be associated with a rear interior portion of the motor vehicle.

In one embodiment, the dashboard 20 may include a ventilation system 30 comprising a side ventilation outlet 1. In this case, side ventilation outlet 1 is disposed in front of passenger seat. In another embodiment, the vent 104 could be a side defroster outlet 2, a center ventilator outlet 3, a floor outlet 4, a front defroster outlet 5, or other vent such as the rear roof in SUVs.

In some cases, the ventilation system 30 may include on or more of a heater unit 6, an air duct 7, a blower motor unit 8, a control lever 9, a defroster nozzle 10, a side demister duct 11, a center ventilator duct 12, a side ventilator duct 13, a side ventilator joint 14 and combinations thereof.

In one embodiment, the ventilation system may further comprise one or more ducts that are configured to deliver air to one or more outlets. However, it should be understood that at least one end of a duct may be attached to a system configured to provide hot, cold, or a mixture of hot and cold air. Examples of such systems include air conditioning systems and heating systems. In an embodiment, ducts may be connected to a heating, air conditioning and ventilation (HVAC) system disposed within the motor vehicle.

In some embodiments, the packaging of various components behind dashboard 20 may prevent ducts from continuing in a straight manner behind outlets. As seen in FIG. 1, the ducts may include curved portions. In some cases, curved portions prevent easy access to all portions of the ventilation system 30.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

What is claimed is:

1. An odor controlling composition comprising an aqueous dispersion medium comprising (a) at least one odor neutralizing agent; (b) at least one adhesion agent; and (c) at least one dispersant.

2. The composition of claim 1, wherein the at least one odor neutralizing agent is selected from the group consisting of zeolites, activated carbons, zinc salts, zinc oxides, cyclodextrins, fatty alcohol esters, aliphatic aldehydes (C10, C11, and C12), zinc ricinoleate actives, benzaldehyde, soyethyl morpholinium ethosulfate, Meelium®, and Metazene®.

3. The composition of claim 1, wherein the at least one odor neutralizing agent is zinc salts selected from the group consisting of zinc borate, zinc caprylate, zinc chloride, zinc ricinoleate, zinc sulfate heptahydrate, zinc undecylenate, and mixtures thereof.

4. The composition of claim 1, wherein the at least one adhesion agent is selected from the group consisting of glycol and polyhydric alcohol.

5. The composition of claim 1, wherein the at least one adhesion agent is a glycol selected from the group consisting of, propylene glycol, dipropylene glycol, butylene glycol, hexylene glycol, polyethylene glycol, and mixtures thereof.

6. The composition of claim 1, wherein the at least one dispersant is selected from the group consisting of butyl ethers, dipropylene glycol methyl ether (DOWANOL™ DPM), D[propylene glycol methyl ether, ethyl alcohol SDA-40, isopropyl alcohol, tropropylene, glycol (mono) methyl ether, alkylene glycol and alkyl polyglycoside.

7. The composition of claim 1, wherein the at least one dispersant is selected from the group consisting of alcohols such as methyl alcohol, ethyl alcohol, n-propyl alcohol, isopropyl alcohol, glycols such as ethylene glycol, diethylene glycol, triethylene glycol, propylene glycol, butylene glycol, hexylene glycol, 1,3-propanediol, 1,4-butanediol, 1,2,4-butanetriol, 1,5-pentanediol, 1,2-hexanediol and 1,6-hexanediol; glycol ethers such as ethylene glycol monomethyl ether and triethylene glycol monoethyl ether; and any combination thereof.

8. The composition of claim 1, wherein the composition further comprises one or more of the following: (d) at least one fragrance; (e) at least one rust inhibitor; (f) at least one surfactant; and (g) at least one compressed gas propellant.

9. The composition according to any one of claims 8, wherein the composition further comprises at least one functional additive.

10. The composition according to claim 10, wherein the at least one functional additive is selected from the group consisting of an oil compound, a deodorizer, an antimicrobial agent, an antiseptic agent and a perfume.

11. A spray pack comprising a spray container device and, packed therein, the composition of any one of claims 1 to 10.
